(19) United States
(12) Patent Application Publication
(10) Pub. No.: US 2017/0338420 A1
(43) Pub. Date: Nov. 23, 2017

(54) ORGANIC COMPOUND AND ORGANIC ELECTROLUMINESCENT DEVICE COMPRISING THE SAME

(71) Applicant: E-RAY OPTOELECTRONICS TECHNOLOGY CO., LTD., Taoyuan City 320 (TW)

(72) Inventors: Heh-Lung Huang, Taoyuan City (TW); Huang-Ming Guo, Taoyuan City (TW); Yen-Jen Lin, Taoyuan City (TW); Teng-Chih Chao, Taoyuan City (TW)

(21) Appl. No.: 15/373,708

(22) Filed: Dec. 9, 2016

(30) Foreign Application Priority Data
May 18, 2016 (TW) 105115287

(51) Int. Cl. H01L 51/00; C07D 235/20; C07D 235/18; H01L 51/50

(52) U.S. Cl. CPC H01L 51/0072; H01L 51/5036; C07D 235/20; H01L 51/0052; C07D 235/18; H01L 51/5012; H01L 51/5016; H01L 51/5072; H01L 51/5076; H01L 51/5092; H01L 51/5096

(57) ABSTRACT

The present disclosure provides a compound represented by formula (I):

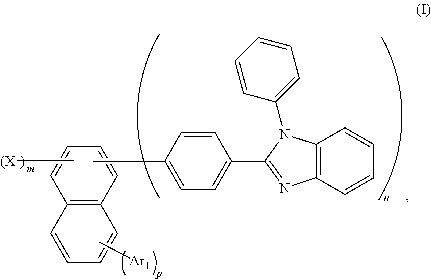

and an organic electroluminescent device including the same.

### BACKGROUND

### 1. Technical Field

The present disclosure relates to compounds for organic electroluminescent devices and organic electroluminescent devices using the same.

### 2. Description of the Related Art

There has been an increasing interest in developing novel organic materials that cater to organic light emitting device (OLED) applications. Such devices are commercially attractive because they offer the cost-advantage in manufacturing high density pixel displays each exhibiting brilliant luminance with a long lifetime, high efficiency, a low driving voltage and a wide color range.

A typical OLED includes at least one organic emissive layer sandwiched between an anode and a cathode. When a current is applied, the anode injects holes, and the cathode injects electrons into the one or more organic emissive layer(s). The injected holes and electrons migrate individually toward the oppositely charged electrode. When an electron and a hole co-localize on the same molecule, an “exciton” is formed, which is a co-localized electron-hole pair having an excited energy state. Light is emitted when the exciton relaxes through a photo emissive mechanism. In order to improve the charge transport capabilities and the luminous efficiency of such devices, one or more additional layers, such as an electron transport layer(s) and/or a hole transport layer(s), or an electron blocking layer(s) and/or a hole block layer(s), have been incorporated around the emissive layer. Doping the host material with another material (i.e., guest) to enhance the performance of the device and to tune the chromaticity has been disclosed in U.S. Pat. Nos. 5,707,745 and 9,153,787, which are incorporated herein by reference in their entirety.

One of the reasons to manufacture an OLED with a multi-layered film structure is the stabilization of the interfaces among the electrodes and the organic layers. In addition, in organic materials, the mobility of the electrons is significantly different from that of holes. Thus, if compatible hole transport layers and electron transport layers are used, holes and electrons can be efficiently transferred to the luminescent layer. Besides, if the densities of the holes and the electrons are balanced in the emitting layer, its luminous efficiency can be increased. Combining the organic layers described above properly can enhance the efficiency and lifetime of the device.

U.S. Pat. Nos. 5,645,948 and 5,766,779 disclose a representative material, 1,3,5-tris(1-phenyl-1H-benzimidazol-2-yl)benzene (TPBI). TPBI has three N-phenyl benzimidazole groups in 1,3,5-substitution sites of benzene, and function both as an electron transporting material and a material emitting blue light. However, TPBI has lower operational stability. Thus, it remains difficult to find an organic material that would satisfy all of the practical requirements of a display.

In addition, U.S. Pat. No. 9,153,787 and US20140284580A1 of E RAY OPTOELECTRONICS TECHNOLOGY CO LTD disclose two series of compounds having the structures of fluoranthene derivatives with imidazole or dibenzothiophene. However, from the current point of view, a device use of the structure of fluoranthene derivatives with imidazole as an electron transport material has a longer lifetime, but needs a higher driving voltage. On the contrary, a device using the structure of fluoranthene derivatives with dibenzothiophene as an electron transport material also needs a higher driving voltage.

Accordingly, there is an urgent need to develop a material for electron transportation in order to extend the lifetime of a device, to improve the luminous efficiency, and to keep a low driving voltage.

### SUMMARY

An object of the present disclosure is to provide a material for an OLED with a high luminous efficiency, a low driving voltage, and a longer lifetime.

The present disclosure provides a compound of formula (I) for an OLED:

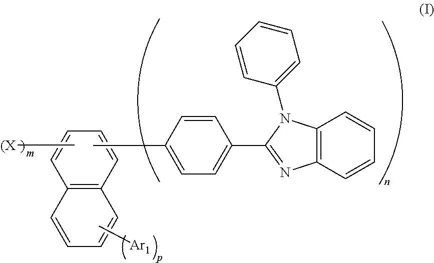

wherein, X represents a substituted or unsubstituted (C1-C10) alkyl, a substituted or unsubstituted (C6-C30) aryl, or a substituted or unsubstituted 5- to 30-membered heteroaryl;

Ar₁ represents a substituted or unsubstituted (C6-C30) aromatic hydrocarbon fused with the naphthalene in formula (I), or a substituted or unsubstituted 5- to 30-membered heteroaromatic hydrocarbon fused with the naphthalene in formula (I);

n is an integer of 1 or 2;

m is an integer of from 0 to 2; and

p is an integer of from 0 to 2.

The present disclosure further provides an organic electroluminescent device, including: a cathode; an anode; and an organic layer disposed between an anode and a cathode, wherein the organic layer includes a compound of the above formula (I).

The organic layer of the organic electroluminescent device of the present disclosure may be an electron transport layer, an electron injection layer, a light-emitting layer, a hole block layer, or an electron block layer. Additionally, besides the organic layer, the organic electroluminescent device may further include at least one layer selected from the group consisting of an electron transport layer, an electron injection layer, a light-emitting layer, a hole block layer, and an electron block layer, other than the organic layer.

According to the present disclosure, using a compound of formula (I) provided by the present disclosure can improve the stability of the device and lower the operational voltage thereof.

### BRIEF DESCRIPTION OF THE DRAWINGS

FIG. 1 is a cross-sectional view illustrating an organic electroluminescent device according to an embodiment of the present disclosure;

FIG. 2 is a cross-sectional view illustrating an organic electroluminescent device according to another embodiment of the present disclosure;

FIG. 3 is a cross-sectional view illustrating an organic electroluminescent device according to yet another embodiment of the present disclosure; and

FIG. 4 shows an electroluminescent spectrum of an organic electroluminescent device according to the present disclosure.

### DETAILED DESCRIPTION OF THE EMBODIMENTS

The following specific embodiments are provided to illustrate the disclosure of the present disclosure, so as to enhance the understanding of the advantages and effects disclosed the specification of the present disclosure by those skilled in the art.

All of the ranges and values disclosed herein can be included and combined. For example, when any value, such as an integer or a point value, falls within the range described herein, a sub-range can be deducted based on the point value or the numerical value as an upper limit or a lower limit. In addition, the groups listed herein, such as groups X and Ar₁ or the substituents thereof, can all be combined in formula (I) with other groups.

According to the present disclosure, the compound for an OLED application is represented by formula (I):

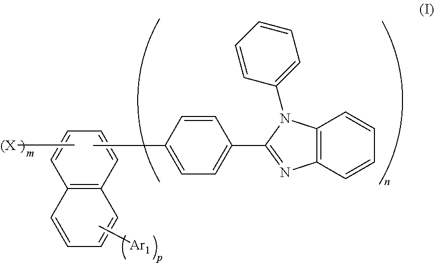

wherein X represents a substituted or unsubstituted (C1-C10) alkyl, a substituted or unsubstituted (C6-C30) aryl, or a substituted or unsubstituted 5- to 30-membered heteroaryl;

Ar₁ represents a substituted or unsubstituted (C6-C30) aromatic hydrocarbon fused with the naphthalene in formula (I) or a substituted or unsubstituted 5- to 30-membered heteroaromatic hydrocarbon fused with the naphthalene in formula (I);

n is an integer of 1 or 2;

m is an integer of from 0 to 2; and

p is an integer of from 0 to 2.

In an embodiment, X represents an unsubstituted (C1-C10) alkyl, an unsubstituted (C6-C30) aryl, or a (C6-C30) aryl substituted by a (C1-C10) alkyl.

In an embodiment, Ar₁ represents an unsubstituted (C6-C30) aromatic hydrocarbon fused with the naphthalene of formula (I), an unsubstituted 5- to 30-membered heteroaromatic hydrocarbon fused with the naphthalene of formula (I) or a 5- to 30-membered heteroaromatic hydrocarbon substituted by a (C6-C30) aryl fused with the naphthalene in formula (I). In an embodiment, the 5- to 30-membered heteroaromatic hydrocarbon or the 5- to 30-membered heteroaryl each independently includes at least one hetero atom selected from the group consisting of N, O and S.

Based on the foregoing, in an embodiment, X represents an unsubstituted (C1-C10) alkyl, an unsubstituted (C6-C30) aryl or a (C6-C30) aryl substituted by a (C1-C10) alkyl, and Ar₁ represents an unsubstituted (C6-C30) aromatic hydrocarbon, an unsubstituted 5- to 30-membered heteroaromatic hydrocarbon, or a 5- to 30-membered heteroaromatic hydrocarbon substituted by a (C6-C30) aryl, wherein the 5- to 30-membered heteroaromatic hydrocarbon includes at least one hetero atom selected from the group consisting of N, O and S, and Ar₁ is fused with the naphthalene in formula (I).

In an embodiment, X represents methyl, phenyl or tolyl.

Moreover, in an embodiment, the substituted or unsubstituted 5- to 30-membered heteroaromatic hydrocarbon is a substituted or unsubstituted 5- to 20-membered heteroaromatic hydrocarbon or a substituted or unsubstituted 5- to 15-membered heteroaromatic hydrocarbon. In an embodiment, Ar₁ represents quinoline, acenaphthene, furan, diphenylfuran, thiophene, pyridine or benzene, and Ar₁ is fused with the naphthalene of formula (I).

Herein, the term “substituted” in the phrase “substituted or unsubstituted” means that the H atom in certain functional group is replaced by another atom or group (i.e. substituent). Each of the substituents can independently be at least one selected from the group consisting of deuterium; halogen; (C1-C30) alkyl; (C1-C30) alkoxy; (C6-C30) aryl; 5- to 30-membered heteroaryl, wherein the 5- to 30-membered heteroaryl can be substituted by a (C6-C30) aryl; 5- to 30-membered heteroaryl substituted by (C6-C30) aryl; (C3-C30) cycloalkyl; 5- to 7-membered heterocycloalkyl; tri(C1-C30)alkylsilyl; tri(C1-C30)arylsilyl; di(C1-C30)alkyl(C6-C30)arylsilyl; (C1-C30)alkyldi(C6-C30)arylsilyl; (C2-C30)alkenyl; (C2-C30)alkynyl; cyano; di(C1-C30)alkylamino; di(C6-C30)arylamino; (C1-C30)alkyl(C6-C30)arylamino; di(C6-C30)arylboryl; di(C1-C30)alkylboryl; (C1-C30)alkyl(C6-C30)arylboryl; (C6-C30)aryl(C1-C30)alkyl; (C1-C30)alkyl(C6-C30)aryl; carboxyl; nitro; and hydroxyl.

Herein, “alkyl” includes methyl, ethyl, n-propyl, isopropyl, n-butyl, isobutyl, tert-butyl, etc.

In an embodiment, the (C1-C10) alkyl is substituted by at least one substituent selected from the group consisting of (C1-C10) alkyl, (C6-C30) aryl, and 5- to 30-membered heteroaryl.

Herein, “aryl” refers to a monocyclic ring or fused ring derived from aromatic hydrocarbon, and includes, for example, phenyl, biphenyl, terphenyl, naphthyl, binaphthyl, phenylnaphthyl, naphthylphenyl, fluorenyl, phenylfluorenyl, benzofluorenyl, dibenzofluorenyl, phenanthrenyl, phenylphenanthrenyl, anthryl, indenyl, triphenylenyl, pyrenyl, naphthacenyl, pyrylo, chrysenyl, naphthonaphthyl, fluoranthenyl and acenaphthenyl.

Herein, “5- to 30-membered heteroaromatic hydrocarbon” is referring to an aromatic hydrocarbon whose main chain has 5 to 30 atoms including at least one hetero atom selected from the group consisting of B, N, O, S, P(═O), Si, and P; is a monocyclic ring, or a fused ring condensed with at least one benzene ring; is partially saturated; is formed by linking at least one heteroaromatic hydrocarbon or aromatic hydrocarbon to a heteroaromatic hydrocarbon through one or more single bonds; and includes monocyclic heteroaromatic hydrocarbon, such as, furan, thiophene, pyrrole, imidazole, pyrazole, thiazole, thiadiazole, isothiazole, isooxazole, oxazole, oxadiazole, triazine, tetrazine, triazole, tetrazole, furazane, pyridine, pyrazine, pyrimidine, and pyridazine; and is a fused heterocyclic aromatic hydrocarbon, such as, benzofuran, benzothiophene, isobenzofuran, dibenzofuran, dibenzothiophene, benzoimidazole, benzothiazole, benzoisothiazole, benzoisooxazole, benzooxazole, isoindole, indole, indazole, benzothiadiazole quinole, isoquinole, cinnoline, quinazoline, quinoxaline, carbazole, phenoxazine, phenanthridine, benzodioxole, and dihydroacridine.

Herein, “5- to 30-membered heteroaryl” is referring to an aryl whose main chain has 5 to 30 atoms including at least one hetero atom selected from the group consisting of B, N, O, S, P(═O), Si, and P; is a monocyclic ring, or a fused ring condensed with at least one benzene ring; is partially saturated; is formed by linking at least one heteroaryl or aryl to a heteroaryl through one or more single bonds; and includes monocyclic heteroaryl, such as, furyl, thienyl, pyrrolyl, imidazolyl, pyrazolyl, thiazolyl, thiadiazolyl, isothiazolyl, isooxazolyl, oxazolyl, oxadiazolyl, triazinyl, tetrazinyl, triazolyl, tetrazolyl, furazanyl, pyridyl, pyrazinyl, pyrimidinyl, and pyridazinyl; and is a fused cyclic heteroaryl, such as, benzofuryl, benzothienyl, isobenzofuryl, dibenzofuryl, dibenzothienyl, benzoimidazolyl, benzothiazolyl, benzoisothiazolyl, benzoisooxazolyl, benzooxazolyl, isoindolyl, indolyl, indazolyl, benzothiadiazolyl, quinolyl, isoquinolyl, cinnolinyl, quinazolinyl, quinoxalinyl, carbazolyl, phenoxazinyl, phenanthridinyl, benzodioxolyl and dihydroacridinyl.

In an embodiment, the (C6-C30)aryl or the (C6-C30)aromatic hydrocarbon is each independently substituted by at least one substituent selected from the group consisting of (C1-C10) alkyl, (C6-C30)aryl and 5- to 30-membered heteroaryl.

In an embodiment, the 5- to 30-membered heteroaryl and the 5- to 30-membered heteroaromatic hydrocarbon are each independently substituted by at least one substituent selected from the group consisting of (C1-C10) alkyl, (C6-C30)aryl and 5- to 30-membered heteroaryl.

In an embodiment, when n is 1, m is 0, and p is 1, Ar₁ represents quinoline, acenaphthene, pyridine, furan, diphenylfuran, or thiophene, the compound of formula (I) is, for example, one selected from the group consisting of

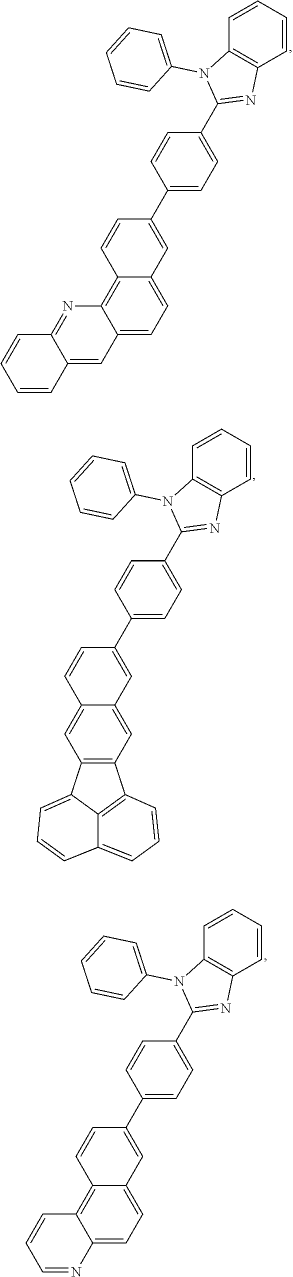 [SMILES: c1ccc(-n2c(-c3ccc(-c4ccc5c(ccc6cc7ccccc7nc65)c4)cc3)nc3ccccc32)cc1 ; c1ccc(-n2c(-c3ccc(-c4ccc5cc6c(cc5c4)-c4cccc5cccc-6c45)cc3)nc3ccccc32)cc1 ; c1ccc(-n2c(-c3ccc(-c4ccc5c(ccc6ncccc65)c4)cc3)nc3ccccc32)cc1]

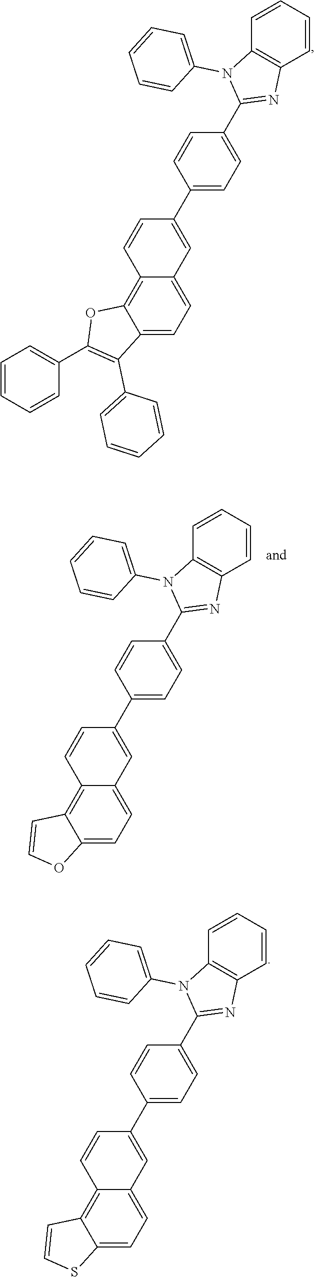 [SMILES: c1ccc(-c2oc3c(ccc4cc(-c5ccc(-c6nc7ccccc7n6-c6ccccc6)cc5)ccc43)c2-c2ccccc2)cc1 ; c1ccc(-n2c(-c3ccc(-c4ccc5c(ccc6occc65)c4)cc3)nc3ccccc32)cc1 ; c1ccc(-n2c(-c3ccc(-c4ccc5c(ccc6sccc65)c4)cc3)nc3ccccc32)cc1]

In an embodiment, when n is 1, m is 0 and p is 2, Ar₁ represents pyridine. For example, the compound of formula (I) is

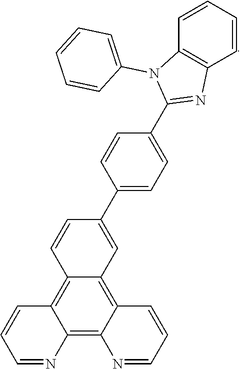 [SMILES: c1ccc(-n2c(-c3ccc(-c4ccc5c(c4)c4cccnc4c4ncccc54)cc3)nc3ccccc32)cc1]

In an embodiment, when n is 1, m is 1 and p is 0, X represents phenyl or tolyl, the compound of formula (I) is

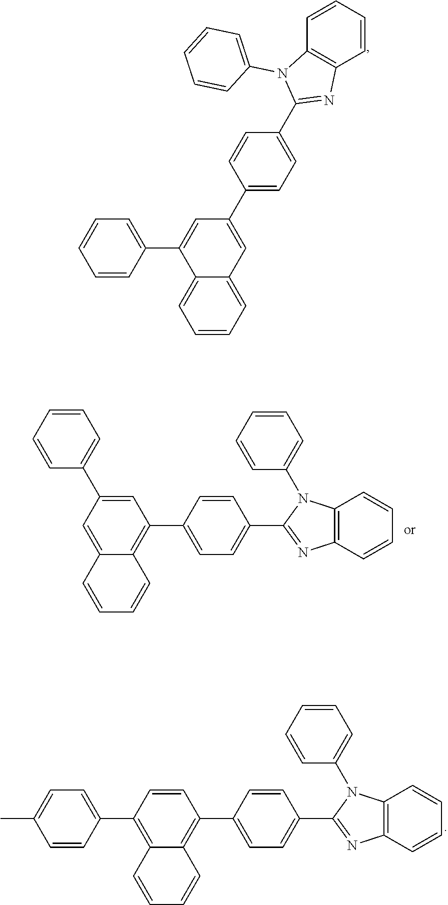 [SMILES: c1ccc(-c2cc(-c3ccc(-c4nc5ccccc5n4-c4ccccc4)cc3)cc3ccccc23)cc1 ; c1ccc(-c2cc(-c3ccc(-c4nc5ccccc5n4-c4ccccc4)cc3)c3ccccc3c2)cc1 ; Cc1ccc(-c2ccc(-c3ccc(-c4nc5ccccc5n4-c4ccccc4)cc3)c3ccccc23)cc1]

In an embodiment, when n is 1, m is 2 and p is 0, X represents phenyl, the compound of formula (I) is

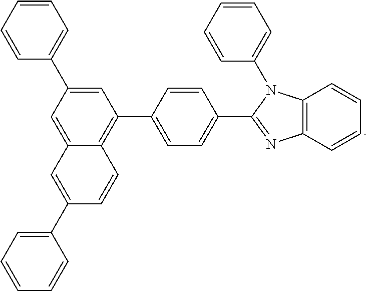 [SMILES: c1ccc(-c2ccc3c(-c4ccc(-c5nc6ccccc6n5-c5ccccc5)cc4)cc(-c4ccccc4)cc3c2)cc1]

In an embodiment, when n is 1, m is 2 and p is 1, X represents phenyl, and Ar₁ represents benzene. For example, the compound of formula (I) is

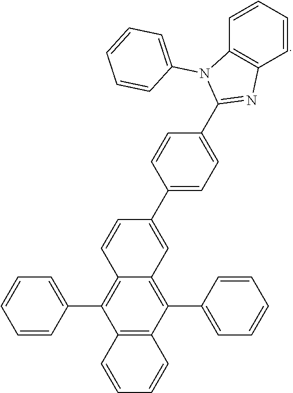 [SMILES: c1ccc(-c2c3ccccc3c(-c3ccccc3)c3cc(-c4ccc(-c5nc6ccccc6n5-c5ccccc5)cc4)ccc23)cc1]

In an embodiment, when n is 2, m is 0 and p is 0, the compound of formula (I) is

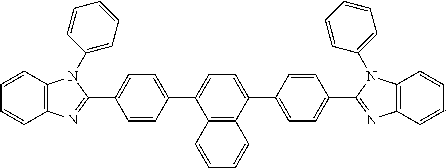 [SMILES: c1ccc(-n2c(-c3ccc(-c4ccc(-c5ccc(-c6nc7ccccc7n6-c6ccccc6)cc5)c5ccccc45)cc3)nc3ccccc32)cc1]

In an embodiment, when n is 2, m is 1 and p is 0, X represents methyl, and the compound of formula (I) is

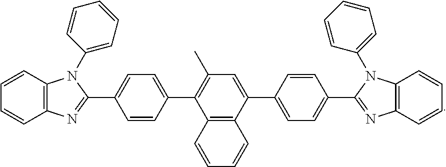 [SMILES: Cc1cc(-c2ccc(-c3nc4ccccc4n3-c3ccccc3)cc2)c2ccccc2c1-c1ccc(-c2nc3ccccc3n2-c2ccccc2)cc1]

In an embodiment, when n is 2, m is 2 and p is 0, X represents methyl, and the compound of formula (I) is

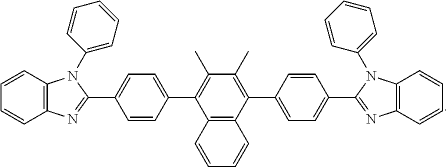 [SMILES: Cc1c(C)c(-c2ccc(-c3nc4ccccc4n3-c3ccccc3)cc2)c2ccccc2c1-c1ccc(-c2nc3ccccc3n2-c2ccccc2)cc1]

The present disclosure further provides an organic electroluminescent device, including a cathode; an anode; and an organic layer disposed between an anode and a cathode, wherein the organic layer includes a compound of the above formula (I).

A series of reactions represented by schemes 1 and 2 below can be performed to synthesize a compound of formula (I), but it is not limited thereto.

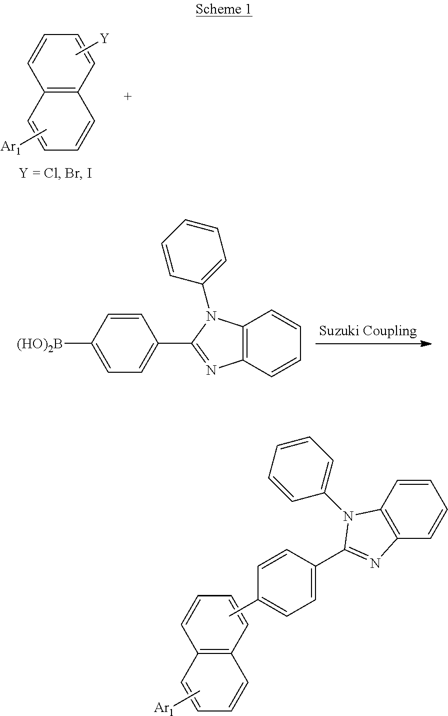

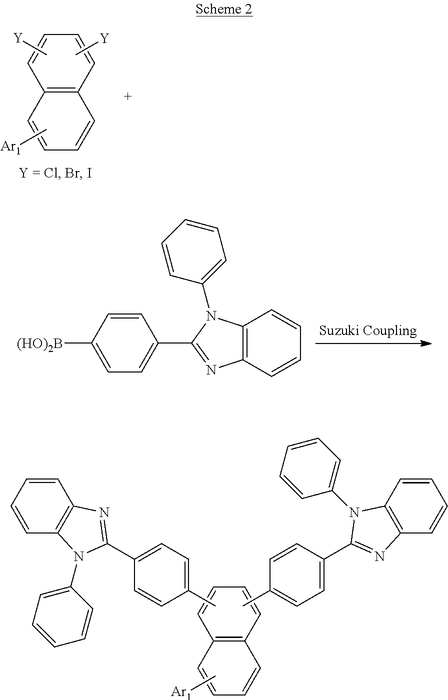

The present disclosure further provides an organic electroluminescent device, including a cathode; an anode; and an organic layer disposed between an anode and a cathode, wherein the organic layer includes a compound of formula (I) of the present disclosure.

The compound of formula (I) can be used for the organic layer of an OLED. Therefore, the OLED of the present disclosure has at least one organic layer interposed between an anode and a cathode on a substrate, wherein the organic layer includes the aforementioned compound of formula (I). The organic layer may be a light-emitting layer, a hole block layer, an electron transport layer, an electron injection layer or a hole transport layer. Besides the organic layer, the organic electroluminescent device can further include at least one layer selected from the group consisting of an electron transport layer, an electron injection layer, a light-emitting layer, a hole block layer, and an electron block layer. The organic layer including the compound of formula (I) may preferably be an electron transport/injection layer, and can be single material of the compound of formula (I), or can be in combination with electrically injecting dopants (n/p type).

Electrically conducting dopants to be used for the electron transport layer are preferably organic alkali/alkaline metal complexes, oxides, halides, carbonates, and phosphates of alkali/alkaline group metals containing at least one metal selected from lithium and cesium. Such organic metal complexes are known in the aforementioned patent documents and elsewhere, and a suitable organic metal complex can be selected from them and used in the present disclosure.

In an embodiment, calculated based on the weight of the organic layer, the content of the compound of formula (I) is in a range of from about 25 wt % to about 100 wt %. Besides, the thickness of the organic layer is in a range of from about 1 nm to about 500 nm.

In another embodiment, the compound of formula (I) can be a single material to form the organic layer, or can be in combination with electrically injecting dopants (n/p type) to form the organic layer. Normally, calculated based on the weight of the organic layer, the content of the electrically injecting dopants is in a range of from about 0 wt % to about 75 wt %. When the compound of formula (I) is in combination with electrically injecting dopants (n/p type) to form the organic layer, for example, an electron transport layer or an electron injection layer, the content of the electrically injecting dopants is in a range of from about 10 wt %, 20 wt % or 25 wt % to about 50 wt %, 60 wt % or 75 wt %.

In an embodiment, the organic layer is a light-emitting layer including a fluorescent or phosphorescent emitter. When the organic layer is not a light-emitting layer, the organic electroluminescent device further includes a light-emitting layer including a fluorescent or phosphorescent emitter and free of the compound represented by formula (I), and the emitting layer is interposed between the anode and the cathode.

In an embodiment, a hole injection layer, a hole transport layer, and a light-emitting layer are further included in an order between the anode and the organic layer, and an electron injection layer is further included between the organic layer and the cathode, and the organic layer is an electron transport layer.

In an embodiment, the organic electroluminescent device emits white light.

The structure of the organic electroluminescent device of the present disclosure is illustrated below with reference to the drawing, but it is not limited thereto.

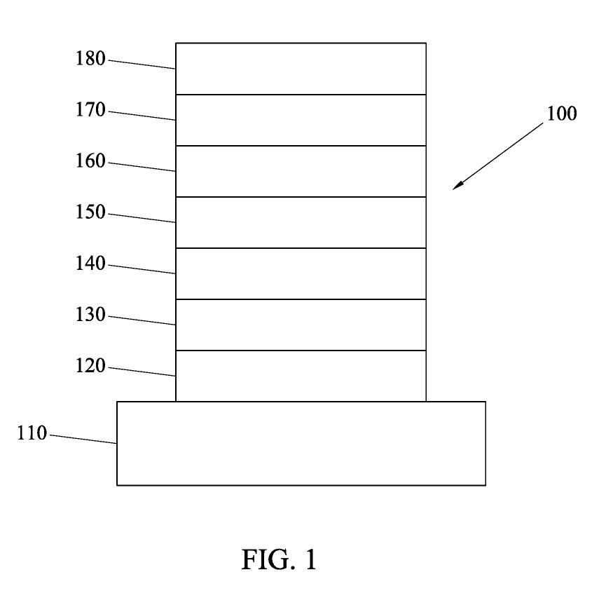

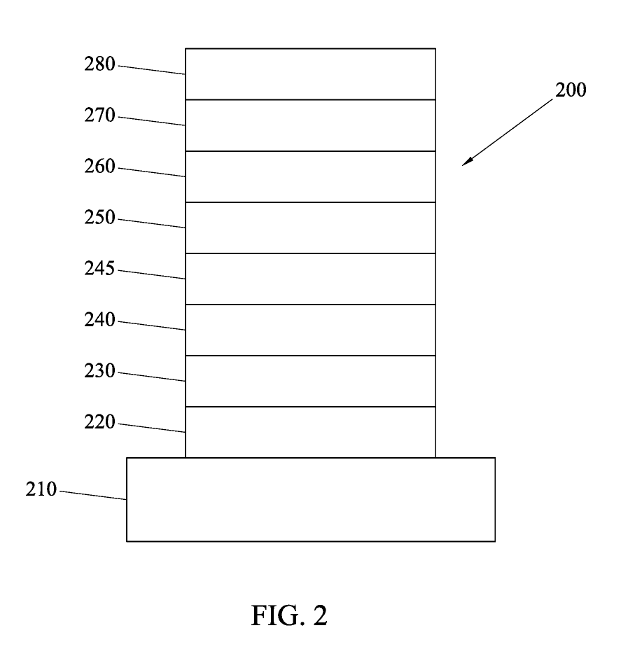

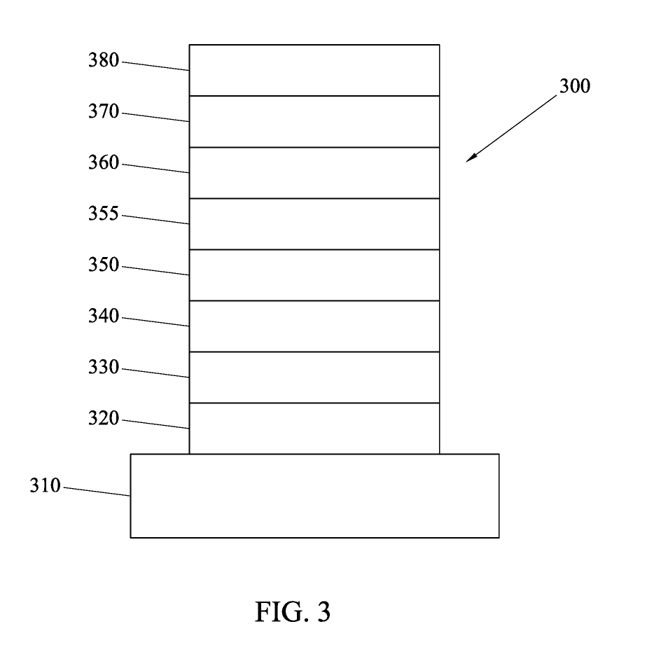

FIG. 1 is a cross-sectional schematic view of an organic electroluminescent device according to an embodiment of the present disclosure. An organic electroluminescent device 100 includes a substrate 110, an anode 120, a hole injection layer 130, a hole transport layer 140, a light-emitting layer 150, an electron transport layer 160, an electron injection layer 170, and a cathode 180. The organic electroluminescent device 100 may be fabricated by depositing the aforementioned layers in an order. FIG. 2 is a cross-sectional schematic view of an organic electroluminescent device according to another embodiment of the present disclosure, which is similar to FIG. 1. Besides a substrate 210, an anode 220, a hole injection layer 230, a hole transport layer 240, a light-emitting layer 250, an electron transport layer 260, an electron injection layer 270 and a cathode 280, the difference is that an exciton block layer 245 of the organic electroluminescent device of FIG. 2 is interposed between the hole transport layer 240 and the light-emitting layer 250. FIG. 3 is a cross-sectional schematic view of an organic electroluminescent device according to another embodiment of the present disclosure, which is also similar to FIG. 1. Besides a substrate 310, an anode 320, a hole injection layer 330, a hole transport layer 340, a light-emitting layer 350, an electron transport layer 360, an electron injection layer 370 and a cathode 380, the difference is that an exciton block layer 355 of the organic electroluminescent device of FIG. 3 is interposed between the light-emitting layer 350 and the electron transport layer 360.

Alternatively, the organic electroluminescent device may be fabricated using the reverse structures of the devices shown in FIGS. 1 to 3. In such reverse structures, one or more layer(s) may be added or omitted as needed.

Materials used for a hole injection layer, a hole transport layer, an electron block layer, a hole block layer, a light-emitting layer, and an electron injection layer may be those conventionally used. For example, an electron-transporting material for forming the electron transport layer differs from the material for forming the emitting layer, and has a property of hole-transporting, so as to facilitate hole mobility in the electron transport layer, and to prevent carrier accumulation due to the difference of dissociation energy between the light-emitting layer and the electron transport layer.

In addition, U.S. Pat. No. 5,844,363 discloses a flexible and transparent substrate in combination with an anode, and the entire disclosure of which is incorporated herein by reference. An example of a p-type doped hole transport layer is m-MTDATA doped with F₄-TCNQ at a molar ratio of 50:1, as disclosed in US Patent Application Publication No. 20030230980A1, and the entire disclosure of which is incorporated herein by reference. An example of an n-type doped electron transport layer is BPhen doped with Li at a molar ratio of 1:1, as disclosed in US Patent Application Publication No. 20030230980A1, and the entire disclosure of which is incorporated herein by reference. The entire disclosures of the exemplary cathodes of U.S. Pat. Nos. 5,703,436 and 5,707,745 are incorporated herein by reference, wherein the cathodes each has a thin layer of metal, such as Mg/Ag (Mg:Ag), with an overlaying transparent, electrically-conductive, sputter-deposited ITO layer. The theory and use of each block layers is described in U.S. Pat. No. 6,097,147 and US Patent Application Publication No. 20030230980, and the entire disclosures of which are incorporated herein by reference. The entire disclosure of the exemplary of the injection layers of US Patent Application Publication No. 20040174116A1, and the protective layers described in the same application are incorporated herein by reference.

Structures and materials not specifically described may also be used in the present disclosure, such as the organic electroluminescent device comprising polymeric materials (PLEDs) disclosed in U.S. Pat. No. 5,247,190, and the entire disclosure of which is incorporated herein by reference. Furthermore, the organic electroluminescent device having a single organic layer or the organic electroluminescent device formed by stacking, as disclosed in U.S. Pat. No. 5,707,745, may be used, and the entire disclosure of which is incorporated herein by reference.

Unless otherwise specified, any layers in the various embodiments may be deposited by any suitable method. For the organic layers, preferred methods include, for example, thermal evaporation and jet printing described in U.S. Pat. Nos. 6,013,982 and 6,087,196, the disclosures of which are incorporated herein by reference in their entirety; organic vapor phase deposition (OVPD) described in U.S. Pat. No. 6,337,102, the disclosure of which is incorporated herein by reference in its entirety; and deposition by organic vapor jet printing (OVJP) described in U.S. patent application Ser. No. 10/233,470, the disclosure of which is incorporated by reference in its entirety. Other suitable deposition methods include spin-coating and other solution-based processes. It is preferable to conduct solution-based processes in an environment containing nitrogen or inert gas. For the other layers, preferred methods include thermal evaporation. Preferred patterning methods include, for example, deposition through a mask followed by cold welding and the processes of patterning and deposition by integrated ink-jet and OVJD described in U.S. Pat. Nos. 6,294,398 and 6,468,819, the disclosures of which are incorporated by reference in their entirety. Certainly, other methods may be used. The materials to be deposited may be modified to be compatible with the particular deposition method.

The compound of formula (I) of the present disclosure may be used to fabricate amorphous thin layers applied to an organic electroluminescent device by vacuum deposition or spin-coating. When the compound is used in any of the organic layers described above, it exhibits a longer lifetime and better thermal stability with high efficiency and a low driving voltage.

An organic electroluminescent device of the present disclosure is applicable to a single device, which is a device with its structure arranged in array, or a device having a cathode and an anode arranged in an X-Y coordinates of the array. The present disclosure can provide an organic electroluminescent device with significantly improved luminous efficiency and driving stability over the conventional devices. Besides, when combining with phosphorescent dopants in the light-emitting layer, the organic electroluminescent device of the present disclosure can perform better and emits white light while applying to full-color or multicolor display panels.

Several properties and effects of the present disclosure will be described in more detail below with reference to the examples. However, these detailed examples are merely used to illustrate the properties of the present disclosure. The present disclosure is not limited by these examples.

### Synthesis Example 1

Into the reaction flask, 1,4-dibromonaphthalene (10 g, 34.69 mmol), 4-(1-phenyl-1H-benzimidazol-2-yl)phenylboronic acid (23.17 g, 76.91 mmol), K₂CO₃ (24.16 g, 157.96 mmol) and Pd(PPh₃)₄ (4.04 g) were added. Then, 150 mL of toluene, 30 mL of ethanol and 60 mL of ddH₂O were added thereto. The mixture was stirred, and refluxed at 80° C. for 16 hours. After the reaction was completed, 100 mL of ddH₂O was added thereto, and the mixture was stirred until cooling to room temperature. The reactant was filtered, the solid residue was obtained, and 250 mL of tetrahydrofuran was added thereto with heating and stirring until the whole solid residue was dissolved. The solution passed through a silica gel column. The eluent was concentrated by distillation, and then ethyl acetate was added to wash, and the solid residue was obtained. After the solid residue was oven-dried, compound A1 was obtained as a white solid (11 g, yield: 47.33%).

¹H NMR (400 MHz, CDCl₃, δ): 7.9-7.93 (m, 4H); 7.71-7.73 (d, 4H); 7.42-7.57 (m, 19H); 7.28-7.3 (t, 5H).

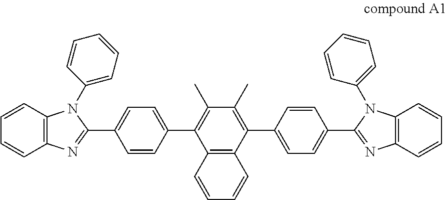 [SMILES: Cc1c(C)c(-c2ccc(-c3nc4ccccc4n3-c3ccccc3)cc2)c2ccccc2c1-c1ccc(-c2nc3ccccc3n2-c2ccccc2)cc1]

### Synthesis Example 2

Into the reaction flask, 1,4-dibromo-2-methyl-naphthalene (10 g, 34.69 mmol), 4-(1-phenyl-1H-benzimidazol-2-yl)phenylboronic acid (23.17 g, 76.91 mmol), K₂CO₃ (24.16 g, 157.96 mmol) and Pd(PPh₃)₄ (4.04 g) were added, and 150 mL of toluene, 30 mL of ethanol and 60 mL of ddH₂O was added thereto. The mixture was stirred, and refluxed at 80° C. for 16 hours. After the reaction was completed, 100 mL of ddH₂O was added thereto, and the mixture was stirred until cooling to room temperature. The reactant was filtered, the solid residue was obtained, and 250 mL of tetrahydrofuran was added thereto with heating and stirring until the whole solid residue was dissolved. The solution was passed through a silica gel column. The eluent was concentrated by distillation, and then ethyl acetate was added to wash, and the solid residue was obtained. After the solid residue was oven-dried, compound A2 was obtained as a white solid (8.5 g, yield: 35.8%).

¹H NMR (400 MHz, CDCl₃, δ): 7.91-7.93 (d, 2H); 7.71-7.73 (m, 4H); 7.15-7.57 (m, 7H); 7.47-7.49 (t, 3H); 7.42-7.44 (m, 5H); 7.34-7.37 (m, 5H); 7.28-7.3 (m, 5H); 2.22 (s, 3H).

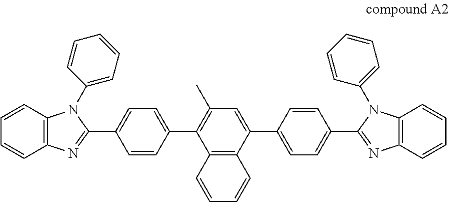 [SMILES: Cc1cc(-c2ccc(-c3nc4ccccc4n3-c3ccccc3)cc2)c2ccccc2c1-c1ccc(-c2nc3ccccc3n2-c2ccccc2)cc1]

### Synthesis Example 3

Into the reaction flask, 1,4-dibromo-2,3-dimethylnaphthalene (10.0 g, 31.85 mmol) and 4-(1-phenyl-1H-benzo[d]imidazol-2-yl)phenylboronic acid (22.01 g, 70.06 mmol) were added. Then, 150 mL of toluene was added thereto. K₂CO₃ (22.01 g, 159.23 mmol) was dissolved in 90 mL of ddH₂O, and added into the reaction flask. Pd(PPh₃)₄ (3.68 g, 3.18 mmol) was added thereto, and followed by the addition of 25 mL of alcohol. The mixture was stirred and refluxed at 80° C. for 16 hours. After the reaction was completed, 200 mL of ddH₂O was added thereto, and the mixture was stirred until cooling to room temperature. The reactant was filtered, the solid residue was obtained, and washed with ddH₂O and toluene. The mixed solution (200 mL of ddH₂O, 50 mL of methanol and 150 mL of toluene) was added to the solid residue, and the mixture was stirred for 30 minutes and filtered for two repetitions. After the solid residue was oven-dried, compound A3 was obtained as a white solid (12.84 g, yield: 58.19%).

¹H NMR (400 MHz, CDCl₃, δ): 7.937-7.916 (d, 2H); 7.733-7.714 (m, 4H); 7.587-7.570 (m, 6H); 7.549-7.508 (m, 6H); 7.446-7.345 (m, 5H); 7.307-7.278 (m, 5H); 7.238-7.224 (m, 2H); 2.256-2.236 (m, 6H).

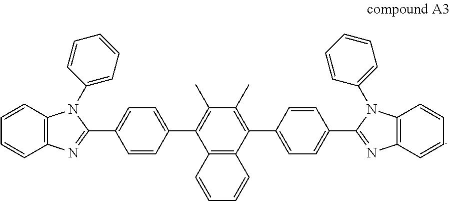 [SMILES: Cc1c(C)c(-c2ccc(-c3nc4ccccc4n3-c3ccccc3)cc2)c2ccccc2c1-c1ccc(-c2nc3ccccc3n2-c2ccccc2)cc1]

### Synthesis Example 4

Into the reaction flask, 2-bromo-9,10-diphenylanthracene (32.76 g, 80 mmol), 4-(1-phenyl-1H-benzimidazol-2-yl)phenylboronic acid (26.48 g, 88 mmol), K₂CO₃ (27.64 g, 200 mmol) and Pd(PPh₃)₄ (4.64 g) were added. Then, 597 mL of toluene, 97 mL of ethanol and 194 mL of ddH₂O was added thereto. The mixture was stirred and refluxed at 80° C. for 16 hours. After the reaction was completed, the mixture was stirred until cooling to room temperature. The aqueous layer was removed, the residue was concentrated, and 80 g of silica and tetrahydrofuran were added thereto. The solution passed through a silica gel column. The product was eluted by toluene. The eluent was concentrated to be viscous, poured into a beaker, stood to precipitate, and the solid residue was obtained. After the solid residue was oven-dried, compound A4 was obtained as a gray solid (30 g, yield: 62.33%).

¹H NMR (400 MHz, CDCl₃, δ): 7.78-7.9 (d, 2H); 7.75-7.77 (d, 1H); 7.68-7.71 (m, 3H); 7.57-7.64 (m, 9H); 7.48-7.52 (m, 9H); 7.34-8-7.36 (m, 5H).

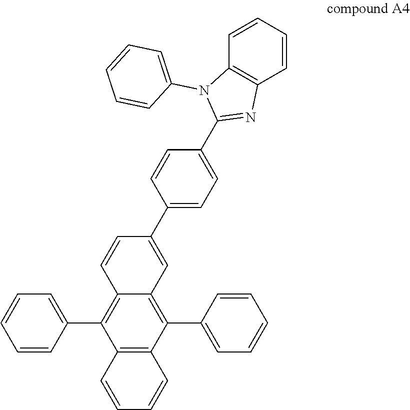 [SMILES: c1ccc(-c2c3ccccc3c(-c3ccccc3)c3cc(-c4ccc(-c5nc6ccccc6n5-c5ccccc5)cc4)ccc23)cc1]

### Example 1 (Fabrication of Organic Electroluminescent Devices)

The substrate was degreased after being cleaned with solvents and UV ozone, before it was loaded into the evaporation system. The substrate was then transferred into a vacuum deposition chamber for deposition of all layers on top of the substrate. By evaporation on a heated boat under a vacuum of approximately 10⁻⁶ Torrs, the following layers were deposited in the following sequence, as shown in FIG. 2:

a) a hole injection layer, 20 nm-thick, HAT-CN;

b) a hole transport layer, 60 nm-thick, HT;

c) an emitting layer, 30 nm-thick, comprising BH doped with 3% of BD by volume (BH and BD are product names from E-ray Optoelectronics Tech Co. Ltd, Taiwan);

d) an electron transport layer, 25 nm-thick, including compound A4 and doped quinolinolato-lithium (Liq);

e) an electron injection layer, 1 nm-thick, lithium fluoride (LiF); and

f) a cathode: approximately 150 nm-thick, including compound A1.

The device structure may be denoted as: ITO/HAT-CN (20 nm)/HT (60 nm)/BH-3% BD (30 nm)/50% compound A4: 50% Liq (25 nm)/LiF (1 nm)/Al (150 nm).

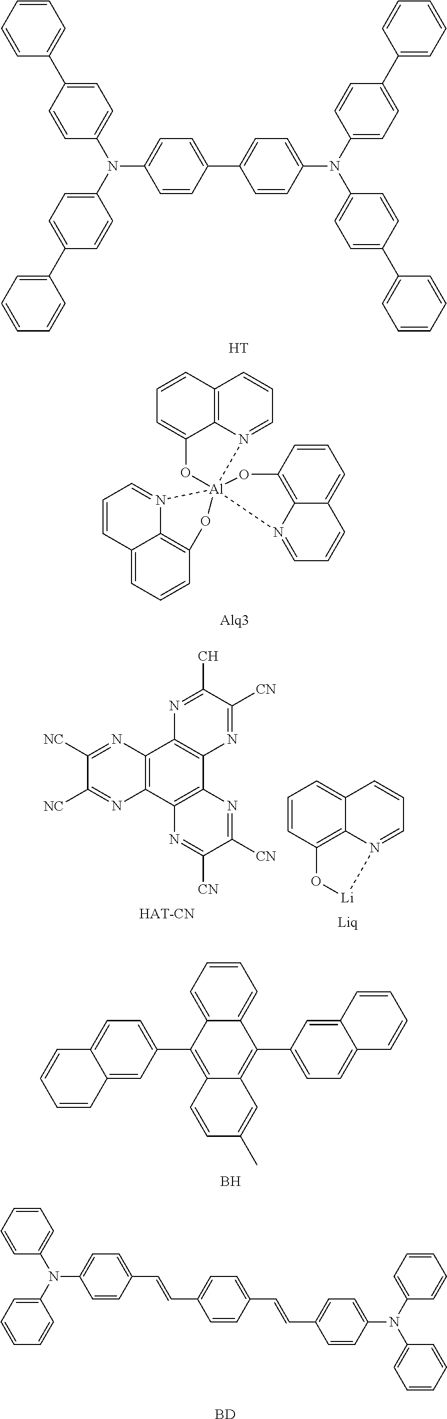

After the deposition of these layers, the device was transferred from the deposition chamber into a dry box for encapsulation, and were subsequently encapsulated using an UV-curable epoxy and a glass lid containing a moisture getter. The organic electroluminescent device has an emission area of 3 mm². The organic electroluminescent device was connected to an outside power source, and was operated under direct current voltage. The characteristics of the emission of light were confirmed and shown in Table 1 below.

The electroluminescent characteristics of all of the fabricated organic electroluminescent devices were evaluated using a constant current source (KEITHLEY 2400 Source Meter, made by Keithley Instruments, Inc., Cleveland, Ohio) and a photometer (PHOTO RESEARCH SpectraScan PR 650, made by Photo Research, Inc., Chatsworth, Calif.) at room temperature.

The operational lifetime (or stability) each of the devices was tested at room temperature and at various initial luminance depending on the color of the light-emitting layer by driving a constant current through the devices. The color was reported using Commission Internationale de l'Eclairage (CIE) coordinates.

### Examples 2 (Fabrication of an Organic Electroluminescent Device)

Except for replacing the 50% Liq of the electron transport layer of Example 1 with 20% Liq, example 2 has the same layer structure as shown in Example 1. The device structure may be denoted as: ITO/HAT-CN (20 nm)/HT (60 nm)/BH-3% BD (30 nm)/80% compound A4: 20% Liq (25 nm)/LiF (1 nm)/Al (150 nm).

### Comparative Example 1 (Fabrication of Organic Electroluminescent Devices)

The organic electroluminescent device of Comparative Example 1 was fabricated similar to the layer structure as shown in Example 1, except that ET was used in place of compound A4 in the electron transport layer of Example 1. The device structure of Comparative Example 1 may be denoted as: ITO/HAT-CN (20 nm)/HT (60 nm)/BH-3% BD (30 nm)/50% ET: 50% Liq (25 nm)/LiF (1 nm)/Al (150 nm).

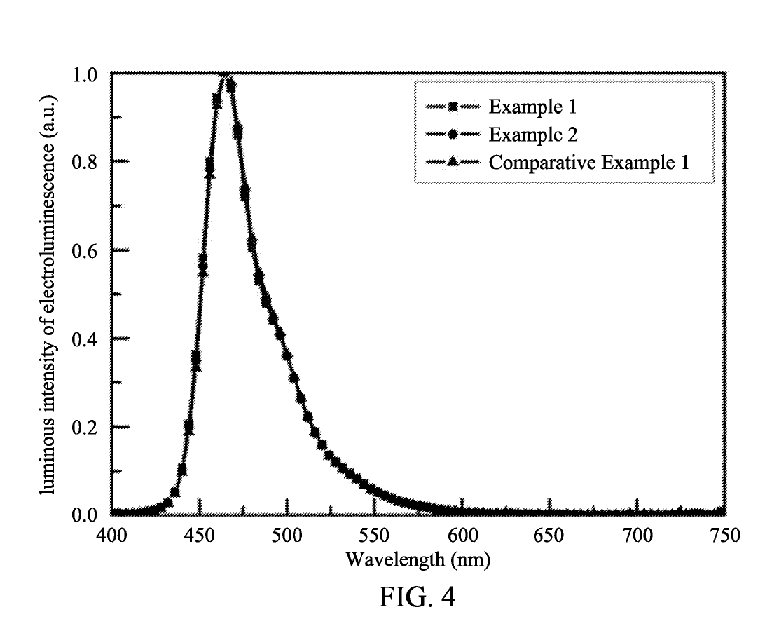

The peak wavelength of the emitted light, maximum luminance efficiency, driving voltage, and stability of the organic electroluminescent devices fabricated in the examples are shown in Table 1. The electroluminescent spectra of the organic electroluminescent device of Example 1 and 2 and Comparative Example 1 are shown in FIG. 4.

TABLE 1

| | | Peak | | | | |
|---|---|---|---|---|---|---|
| | | wavelength | | Maximum | | |
| | | of the | | luminance | | |
| | Compound | emitted | Driving | efficiency | Power | Stability T⁹⁸ |
| | (concentration | light | voltage | (cd/A) @ | efficiency | (hr) @L₀ = |
| | %) | (nm) | (V) | 10 mA/cm² | (lm/W) | 1000 units |
| Example 1 | compound | 464 | 4.56 | 10.39 | 7.16 | 15 |
| | A4 (50) | | | | | |
| Example 2 | compound | 464 | 4.90 | 8.25 | 5.29 | 30 |
| | A4 (80) | | | | | |
| Comparative | ET (50) | 464 | 4.86 | 9.68 | 6.25 | 15 |
| Example 1 | | | | | | |

The present disclosure is not be limited by the above described embodiments, method and examples, but based on all of the embodiments and methods within the scope and spirit of the present disclosure as claimed.

### APPLICABILITY

As described above in detail, the organic electroluminescent device of the present disclosure including the material for the electroluminescent device can achieve long lifetime, has high luminous efficiency, and remains the need for a low driving voltage. Therefore, the organic electroluminescent device of the present disclosure is applicable to flat panel displays, mobile phone displays, light sources utilizing the characteristics of planar light emitters, sign-boards, such that it has a high technical value.

1. A compound represented by formula (I): 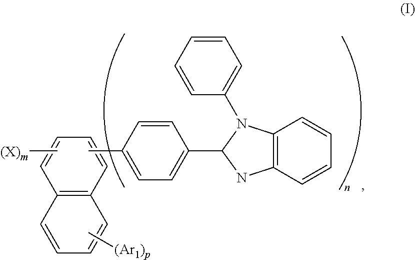
wherein:
X represents a substituted or unsubstituted (C1-C10) alkyl, a substituted or unsubstituted (C6-C30) aryl, or a substituted or unsubstituted 5- to 30-membered heteroaryl;
Ar₁ represents quinoline, acenaphthene, furan, diphenylfuran, thiophene, pyridine or benzene fused with the naphthalene in formula (I);
n is an integer of 1 or 2;
m is an integer of from 0 to 2; and
p is an integer of from 0 to 2, provided that
the compound of formula (I) is 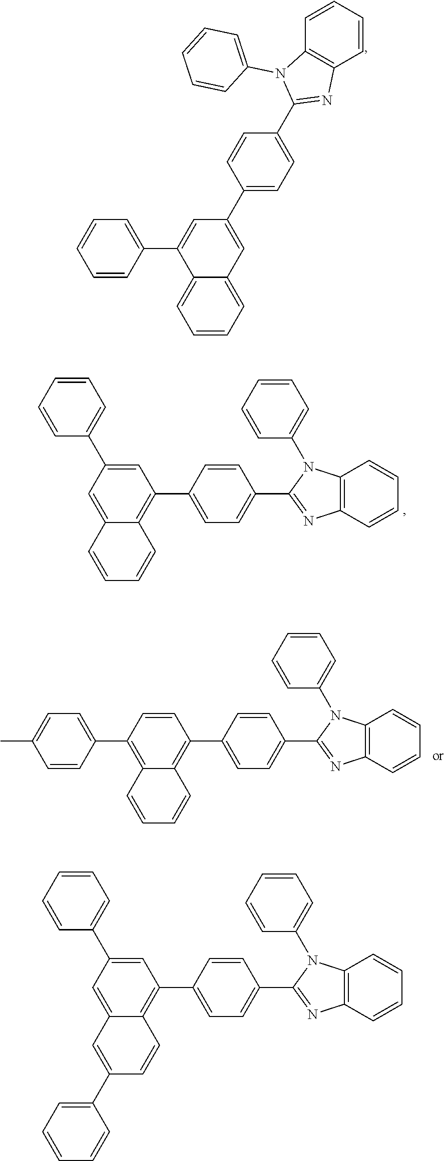 [SMILES: c1ccc(-c2cc(-c3ccc(-c4nc5ccccc5n4-c4ccccc4)cc3)cc3ccccc23)cc1 ; c1ccc(-c2cc(-c3ccc(-c4nc5ccccc5n4-c4ccccc4)cc3)c3ccccc3c2)cc1 ; Cc1ccc(-c2ccc(-c3ccc(-c4nc5ccccc5n4-c4ccccc4)cc3)c3ccccc23)cc1 ; c1ccc(-c2ccc3c(-c4ccc(-c5nc6ccccc6n5-c5ccccc5)cc4)cc(-c4ccccc4)cc3c2)cc1]
when p is 0;
n is 2 when p is 0;
m is 0 when Ar₁ represents quinoline; and
n is 1 when Ar₁ represents acenaphthene or pyridine.

2. The compound of claim 1, wherein X represents an unsubstituted (C1-C10) alkyl, an unsubstituted (C6-C30) aryl or a (C6-C30) aryl substituted by a (C1-C10) alkyl.

3. The compound of claim 1, wherein X represents methyl, phenyl or tolyl.

4. The compound of claim 1, being one selected from the group consisting of

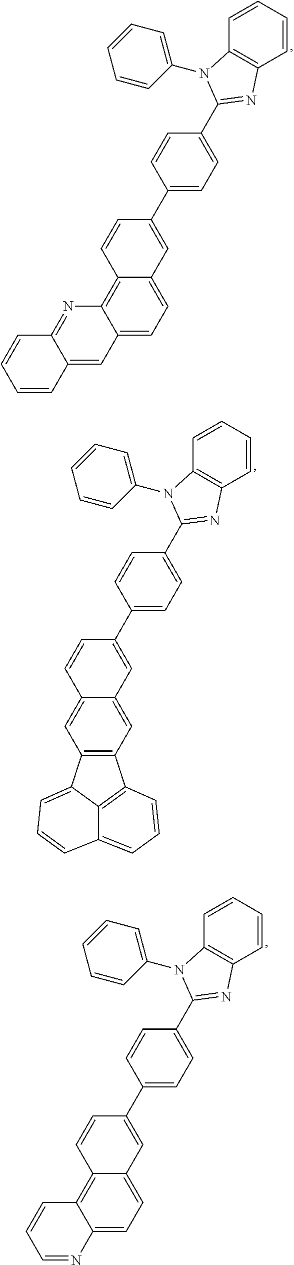 [SMILES: c1ccc(-n2c(-c3ccc(-c4ccc5c(ccc6cc7ccccc7nc65)c4)cc3)nc3ccccc32)cc1 ; c1ccc(-n2c(-c3ccc(-c4ccc5cc6c(cc5c4)-c4cccc5cccc-6c45)cc3)nc3ccccc32)cc1 ; c1ccc(-n2c(-c3ccc(-c4ccc5c(ccc6ncccc65)c4)cc3)nc3ccccc32)cc1]

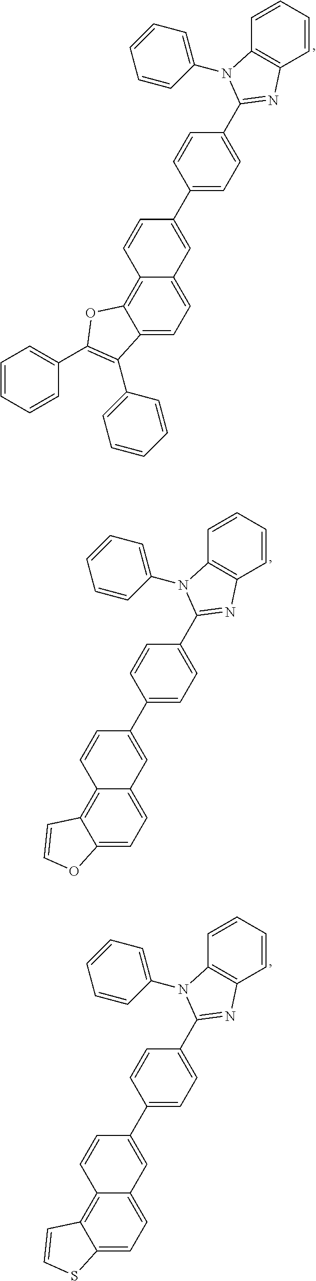 [SMILES: c1ccc(-c2oc3c(ccc4cc(-c5ccc(-c6nc7ccccc7n6-c6ccccc6)cc5)ccc43)c2-c2ccccc2)cc1 ; c1ccc(-n2c(-c3ccc(-c4ccc5c(ccc6occc65)c4)cc3)nc3ccccc32)cc1 ; c1ccc(-n2c(-c3ccc(-c4ccc5c(ccc6sccc65)c4)cc3)nc3ccccc32)cc1]

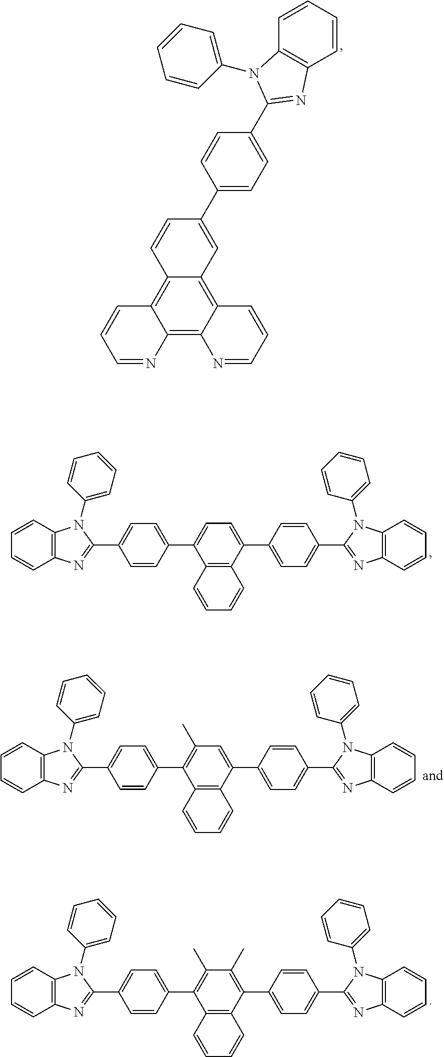 [SMILES: c1ccc(-n2c(-c3ccc(-c4ccc5c(c4)c4cccnc4c4ncccc54)cc3)nc3ccccc32)cc1 ; c1ccc(-n2c(-c3ccc(-c4ccc(-c5ccc(-c6nc7ccccc7n6-c6ccccc6)cc5)c5ccccc45)cc3)nc3ccccc32)cc1 ; Cc1cc(-c2ccc(-c3nc4ccccc4n3-c3ccccc3)cc2)c2ccccc2c1-c1ccc(-c2nc3ccccc3n2-c2ccccc2)cc1 ; Cc1c(C)c(-c2ccc(-c3nc4ccccc4n3-c3ccccc3)cc2)c2ccccc2c1-c1ccc(-c2nc3ccccc3n2-c2ccccc2)cc1]

5. An organic electroluminescent device, comprising:
a cathode;
an anode; and
an organic layer formed between the cathode and the anode, wherein the organic layer comprises the compound according to claim 1.

6. The organic electroluminescent device of claim 5, wherein, based on a total weight of the organic layer, an amount of the compound represented by formula (I) is in a range of from about 25 wt % to about 100 wt %.

7. The organic electroluminescent device of claim 5, wherein a thickness of the organic layer is in a range of from about 1 nm to about 500 nm.

8. The organic electroluminescent device of claim 5, wherein the organic layer is an electron transport layer, an electron injection layer, a light-emitting layer, a hole block layer or an electron block layer.

9. The organic electroluminescent device of claim 5, the organic layer is an electron transport or injection layer comprising n-type electrically conducting dopants.

10. The organic electroluminescent device of claim 9, wherein an amount of the n-type electrically conducting dopants is from about 20 wt % to about 75 wt % based on a total weight of the organic layer.

11. The organic electroluminescent device of claim 5, wherein the organic layer is a light-emitting layer comprising a fluorescent or phosphorescent emitter.

12. The organic electroluminescent device of claim 5, further comprising an emitting layer formed between the cathode and the anode, wherein the emitting layer comprises a fluorescent or phosphorescent emitter and is free of the compound represented by formula (I).

13. The organic electroluminescent device of claim 5, being configured to emit white light.
